(19) United States
(12) Patent Application Publication
(10) Pub. No.: US 2022/0022909 A1
(43) Pub. Date: Jan. 27, 2022

(54) METHOD AND DEVICE FOR THE PERCUTANEOUS RELEASE OF THE A1 PULLEY IN THE HAND

(71) Applicants: Robert E. LINS, Boca Raton, FL (US); Dennis TY, Franklin, TN (US)

(72) Inventors: Robert E. LINS, Boca Raton, FL (US); Dennis TY, Franklin, TN (US)

(21) Appl. No.: 17/381,241

(22) Filed: Jul. 21, 2021

Related U.S. Application Data
(60) Provisional application No. 63/054,278, filed on Jul. 21, 2020.

(51) Int. Cl. A61B 17/3205

(52) U.S. Cl. CPC A61B 17/3205

(57) ABSTRACT

A method and device for the percutaneous release of the A1 pulley in the hand, as the triggering of the associated tendon can occur at the A1 pulley in the hand. A blade/hook construct is provided to release the A1 pulley and subsequently confirm the release. The blade and hook are disposed about opposite ends of an elongated shaft, allowing the tool to be used for both cutting and confirming the release of the A1 pulley in the hand. The blade assembly is adapted to avoid damage to adjacent structures, such as nerves and arteries. Advantageously, the A1 pulley may be cut in a distal-to-proximal or proximal-to-distal manner and the percutaneous skin incision may be made centered over the mid-portion of the A1 pulley, or it may be made more proximal or distal.

### CROSS-REFERENCE TO RELATED APPLICATION

The present patent application/patent claims the benefit of priority of co-pending U.S. Provisional Patent Application No. 63/054,278, filed on Jul. 21, 2020, and entitled “METHOD AND DEVICE FOR THE PERCUTANEOUS RELEASE OF THE A1 PULLEY IN THE HAND” the contents of which are incorporated in full by reference herein.

### FIELD OF THE INVENTION

The present invention relates generally to a surgical tool and the method of use thereof. More specifically, the present invention relates to a surgical tool and method that is used to percutaneously release the A1 pulley in the hand and subsequently confirm the complete release of the A1 pulley in the hand to allow the flexor tendons to move more freely.

### BACKGROUND OF THE INVENTION

Percutaneous A1 pulley release is a widely used procedure to treat the condition known as trigger finger. This procedure is used alternatively to open surgical release of the A1 pulley and injection of local steroids for the treatment of trigger finger. Current methods of percutaneous A1 pulley release involve the use of a large gauge needle to perform the incision which results in a high percentage of incomplete releases of the A1 pulley. No single conventional surgical tool exists that allows the surgeon to both perform and confirm the release of the A1 pulley. The present disclosures provide such a surgical tool and method of use thereof.

### SUMMARY OF THE INVENTION

In various exemplary embodiments, the present disclosure provides a method and device for the percutaneous release of the A1 pulley in the hand, as the triggering of the associated tendon can occur at the A1 pulley in the hand. A blade/hook construct is provided to release the A1 pulley and subsequently confirm the release. The blade assembly is adapted to avoid damage to adjacent structures, such as nerves and arteries. The distal tip of the blade assembly comes to a point to help engage the A1 pulley when cutting is initiated. The A1 pulley may be cut in a distal-to-proximal or proximal-to-distal manner. the percutaneous skin incision may be made centered over the mid-portion of the A1 pulley, or it may be made more proximal or distal.

In one exemplary embodiment, the present invention provides a surgical tool, including: an elongated shaft; a blade assembly coupled to a first end of the elongated shaft; and a hook coupled to a second end of the elongated shaft. The blade assembly includes a cutting edge and a protective spine disposed on opposite sides of said blade assembly. The cutting edge further includes an elongated blade tip protruding from a distal end of the cutting edge. The hook is tapered from the elongated shaft and extends perpendicular to the elongated shaft, terminating in a rounded end to protect anatomical structures. Optionally, the blade assembly includes a cutting edge, a protective spine, and a blade cover. The blade cover includes a raised toggle and a recess that conforms to the cutting edge and protective spine, such that the blade cover is secured to the blade assembly and may slide linearly along the length of the blade assembly to selectively allow the cutting edge to be exposed. Optionally, the hook is tapered from the elongated shaft, coming to a rounded end to protect anatomical structures.

In another exemplary embodiment, the present invention provides a method for using a surgical tool including: making an incision in skin centered over the middle of an A1 pulley in a finger; inserting a blade assembly of a surgical tool into the incision; engaging a distal end of the A1 pulley with a blade tip extending from a distal end of a cutting edge fixed to the blade assembly; translating the blade assembly proximally to cut the A1 pulley; removing the blade assembly from the incision; inserting a hook of the surgical tool to engage the distal end of the A1 pulley; and translating the hook proximally as to confirm the complete release of the A1 pulley. Optionally, the incision is made proximal or distal to the A1 pulley. Alternatively, the blade tip engages a proximal end of the A1 pulley, and the blade assembly is rotated distally to cut the A1 pulley. Alternatively, the hook engages a proximal end of the A1 pulley, and the hook is translated distally as to confirm the complete release of the A1 pulley.

In another exemplary embodiment, the present invention provides a method for using a surgical tool including: making an incision in the skin centered over the middle of an A1 pulley in a finger; inserting a blade assembly of the surgical tool into the small incision with a blade cover in its extended position as to cover a cutting edge; sweeping the covered blade back and forth to push aside soft tissue that may be on top of the A1 pulley to allow subsequent cutting of the A1 pulley; retracting said blade cover as to expose the cutting edge of the blade assembly to allow subsequent cutting of the A1 pulley; engaging a distal end of the A1 pulley with the cutting edge of the blade assembly; translating the cutting edge proximally to cut the A1 pulley; removing the blade assembly from the incision; inserting a hook end of the surgical tool to engage the distal end of the A1 pulley; and translating the hook proximally as to confirm the complete release of the A1 pulley. Optionally, the incision is made proximal or distal to the A1 pulley. Alternatively, the cutting edge engages a proximal end of the A1 pulley and, the cutting edge is translated distally to cut the A1 pulley. Alternatively, the hook engages a proximal end of the A1 pulley and, the hook is translated distally as to confirm the complete release of the A1 pulley.

### BRIEF DESCRIPTION OF THE DRAWINGS

The present disclosure is illustrated and described with reference to the various drawings, in which:

FIG. 1 provides illustration/description of the anatomy of a finger calling attention to the A1 pulley;

FIG. 2 provides a side view of the device described in one embodiment of the present disclosure;

FIG. 3 provides a cross section view of the blade in one embodiment of the present disclosure;

FIG. 4 provides a front prospective view of the device described in one embodiment of the present disclosure with the blade extended;

FIG. 5 provides a front prospective view of the device described in one embodiment of the present disclosure with the blade retracted;

FIG. 6 provides a partial front prospective view of the device described in one embodiment of the present disclosure with the blade extended;

FIG. 7 provides a rear prospective view of the device described in one embodiment of the present disclosure;

FIG. 8 provides a partial rear prospective view of the device described in one embodiment of the present disclosure;

FIG. 9 provides a side view of the device described in one embodiment of the present disclosure with the blade extended;

FIG. 10 provides a side view of the device described in one embodiment of the present disclosure with the blade retracted;

FIG. 11 provides an illustration demonstrating the blade end placement and motion of the device to release the A1 pulley;

FIG. 12 provides an illustration demonstrating the hook end placement and motion to confirm the release of the A1 pulley;

FIG. 13 provides a flow chart demonstrating the method of use of one of the embodiments of the present disclosure; and

FIG. 14 provides a flow chart demonstrating the method of use of another one of the embodiments of the present disclosure.

### DETAILED DESCRIPTION OF THE INVENTION

Again, in various exemplary embodiments, the present disclosure provides a method and device for the percutaneous release of the A1 pulley in the hand, as the triggering of the associated tendon can occur at the A1 pulley in the hand. A blade/hook construct is provided to release the A1 pulley and subsequently confirm the release. The blade assembly is adapted to avoid damage to adjacent structures, such as nerves and arteries. The distal tip of the blade assembly comes to a point to help engage the A1 pulley when cutting is initiated. The A1 pulley may be cut in a distal-to-proximal or proximal-to-distal manner. the percutaneous skin incision may be made centered over the mid-portion of the A1 pulley, or it may be made more proximal or distal.

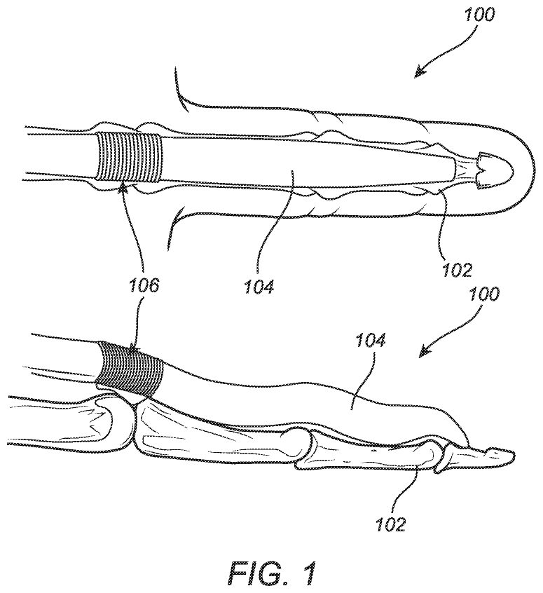

Referring now specifically to FIG. 1, the illustration shows the anatomy of a human finger 100. Depicted in this illustration is a cross section highlighting the bones 102 of a finger 100, a flexor tendon 104, and an A1 pulley 106. This flexor tendon 104 is disposed atop the finger bones 102 and drives the movement of the finger 100. The flexor tendon 104 is supported and aligned by the first annular (A1) pulley 106 which serves as a very important structure of the hand. The A1 pulley 106 acts as a sheath for the flexor tendon 104 and provides biomechanical support as the flexor tendon 104 is activated. The present disclosure provides a surgical tool that is used to percutaneously release this A1 pulley 106 as a treatment for trigger finger.

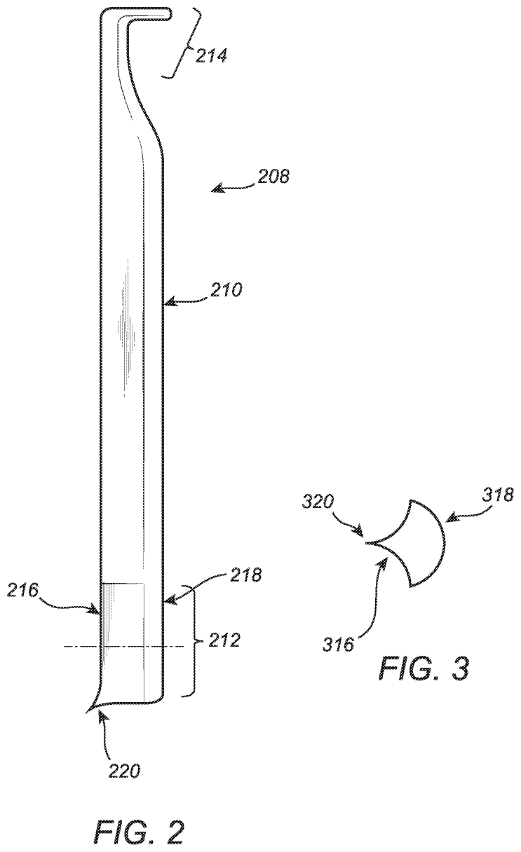

Referring now specifically to FIG. 2, in the first exemplary embodiment, the surgical tool 208 of the present disclosure includes an elongated shaft 210, a blade 212, and a hook 214. The elongated shaft 210 is adapted to be ergonomically shaped as to allow a person to hold the surgical tool 208 while performing its intended task. This elongated shaft 210 can have many cross-sectional shapes including circular, flat, or may have a handle that is enlarged to make the member easier to grab in addition to other handle shapes known to one of ordinary skill in the art. This elongated shaft 210 may also be manufactured to any length that allows the surgical tool 208 to be used comfortably. The elongated shaft 210 comprises of two ends, the blade 212 and hook 214, both fixed to opposite ends of the elongated shaft 210. In this embodiment, the blade 212 is fixedly attached to the blade end of the elongated shaft 210. The blade 212 includes a cutting edge 216 and a protective spine 218 and is shown in greater detail in FIG. 3. The spine 218 is curved to protect surrounding anatomical structures such as nerves and arteries while the tool is being used. A blade tip 220 is positioned on the distal tip of the cutting edge 216 and comes to an elongated point to help engage the A1 pulley 106 when starting to cut. The hook 214 is fixed to the opposite end of the elongated shaft 210. This hook 214 is tapered from the elongated shaft 210 and bends perpendicular to the elongated shaft 210. The hook end is rounded as to allow it to glide across the top of the flexor tendon without causing damage after the A1 pulley is severed, allowing it to be used to confirm the complete release of the A1 pulley.

Referring now specifically to FIG. 3, in the first exemplary embodiment, the blade assembly is made up of a cutting edge 316, blade tip 320, and a protective spine 318. In this embodiment, the spine 318 is an extrusion of the elongated shaft 310 and mimics the curved geometry of the handle to protect surrounding anatomical structures such as nerves and arteries while the tool is being used. The blade tip 320 is tapered from the spine 318 and is extended from the rest of the cutting edge 316 to form a sharp point. Again, the blade tip 320 is positioned on the distal end of the cutting edge 316 and comes to an extended point to help engage the A1 pulley 106 when cutting is initiated.

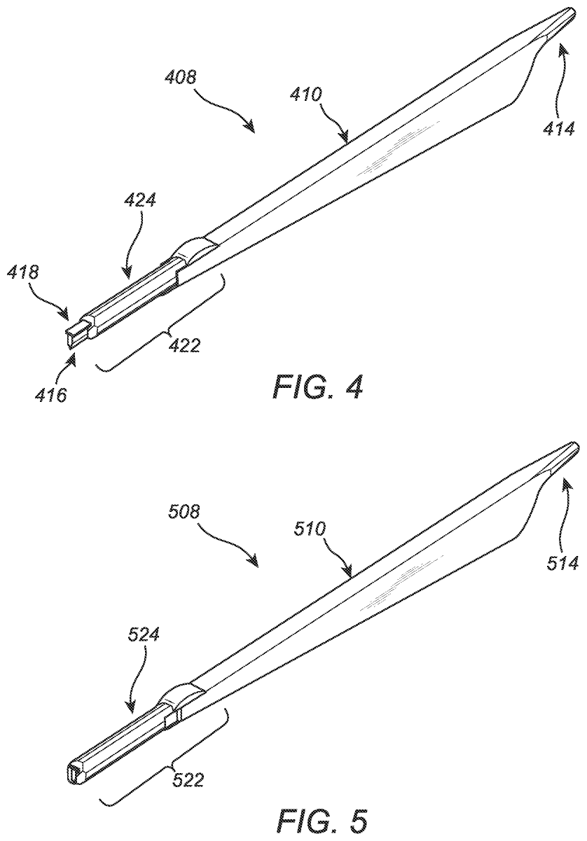

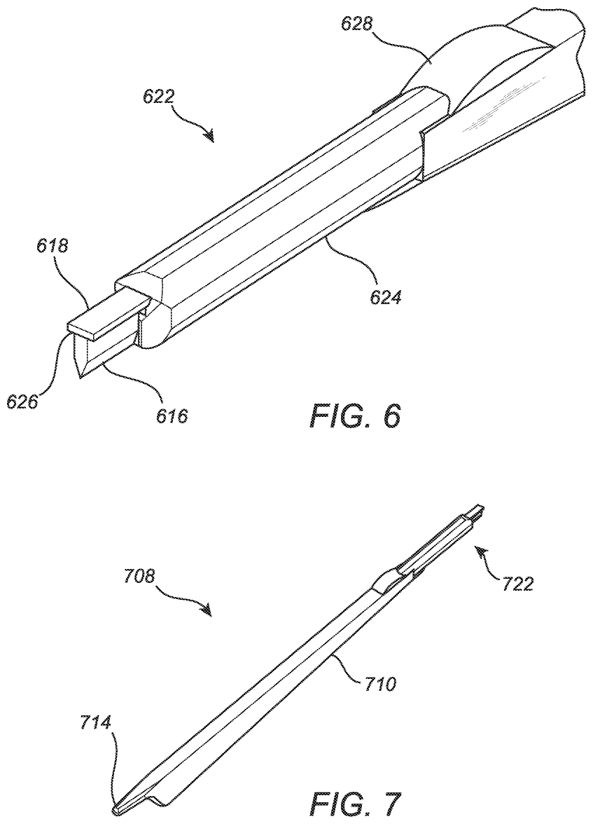

Referring now specifically to FIG. 4, in the second exemplary embodiment, the surgical tool 408 of the present disclosure includes an elongated shaft 410, a blade assembly 422, and a hook 414. The elongated shaft 410 is adapted to be ergonomically shaped as to allow a person to hold the surgical tool 408 while performing its intended task. This elongated shaft 410 can have many cross-sectional shapes including circular, flat, or may have a handle that is enlarged to make the member easier to grab in addition to other handle shapes known to one of ordinary skill in the art. This elongated shaft 410 may also be manufactured to any length that allows the surgical tool 408 to be used comfortably. The elongated shaft 410 comprises of two ends, the blade assembly 422 and hook 414, both fixed to opposite ends of the elongated shaft 410. In this embodiment, the blade assembly 422 includes a cutting edge 416, a protective spine 418, and a blade cover 424. The blade assembly is shown in greater detail in FIG. 6. The spine 418 is a flat portion that extends laterally from the top of the cutting edge 416 to form a T shape and continues along the length of the blade assembly 422 to both secure the blade cover 424 and protect surrounding anatomical structures such as nerves and arteries while the tool is being used. The cutting edge 416 is tapered from the blade assembly 422 thereby creating a sharp edge to perform the cutting of the A1 pulley 106. The blade cover 424 is disposed around the protective spine 418 and cutting edge 416 and includes a recess that conforms to the cutting edge 416 and protective spine 418, such that the blade cover 424 is secured to the blade assembly and may slide along the length of the blade assembly 422 to selectively choose an extended or retracted position when shifted by the user. The hook 414 is fixed to the opposite end of the elongated shaft 410. This hook 414 is tapered from the elongated shaft 410 and the hook end is rounded as to allow it to glide across the top of the flexor tendon without causing damage after the A1 pulley is severed, allowing it to be used to confirm the complete release of the A1 pulley.

Referring now specifically to FIG. 5, in the second exemplary embodiment, the surgical tool 508 of the present disclosure again includes an elongated shaft 510, a blade assembly 522, and a hook 514. The elongated shaft 510 again is made up of two ends, the blade assembly 522 and hook 514, both fixed to opposite ends of the elongated shaft 510. In this embodiment, the blade assembly 522 again includes a cutting edge 416, a protective spine 418, and a blade cover 524. The blade assembly is shown in greater detail in FIG. 6. As before, the blade cover 524 includes a recess that conforms to the cutting edge 416 and protective spine 418, such that the blade cover 524 may slide along the length of the blade assembly 522 when shifted by the user. The blade cover 524 is limited in linear motion by the structure of the elongated shaft or any other type and/or number of mechanisms, such as detents or the like, may also be used to selectively grade and retain the motion of the blade cover 524 atop the blade assembly 522. In this illustrative embodiment the blade cover 524 is illustrated in its extended position to show how it covers the blade assembly. When the blade cover 524 is fully extended, the entire blade assembly 522 is hidden inside the blade cover 524, thus protecting the internal structures of the finger from the cutting edge 416.

Referring now specifically to FIG. 6, in the second exemplary embodiment, the blade assembly 622 is shown in greater detail and again includes a cutting edge 616, a protective spine 618, and a blade cover 624. The spine 618 is a flat portion that extends from the top of the cutting edge 616 to form a T shape and continues along the length of the blade assembly 622 to both secure the blade cover 624 and protect surrounding tissue. This spine 618 extends laterally past the thickness of the cutting edge 616 and creates a notch 626 which aligns the blade cover 624 and keeps it from lifting off the blade assembly 622. This spine 618 is also designed to create a buffer between the cutting edge 616 and any surrounding anatomical structures such as nerves and arteries that may alternatively be damaged by the cutting edge 616. This cutting edge 616 is tapered from the blade assembly 622 thereby creating a sharp edge to perform the cutting of the A1 pulley 106. The blade cover 624 is disposed around the cutting edge 616 and the protective spine 618 and includes a recess that conforms to the cutting edge 616 and protective spine 618, such that the blade cover 624 may slide along the length of the blade assembly 622 when shifted by the user and is kept from lifting off of the blade assembly 622 by way of the notches 626. The blade cover 624 also includes a raised toggle 628 which allows the user to selectively push and pull the blade cover 624 into the desired extended or retracted position. The blade cover 624 is limited in linear motion by the structure of the elongated shaft or any other type and/or number of mechanisms, such as detents or the like, may also be used to selectively grade and retain the motion of the blade cover 624 atop the blade assembly 622. When the blade cover 624 is fully retracted, the cutting edge 616 is exposed, thus allowing the tool to be used for its intended purpose.

Referring now specifically to FIG. 7, in the second exemplary embodiment, the surgical tool 708 of the present disclosure again includes an elongated shaft 710, a blade assembly 722, and a hook 714. The elongated shaft 710 again is made up of two ends, the blade assembly 722 and hook 714, both fixed to opposite ends of the elongated shaft 710.

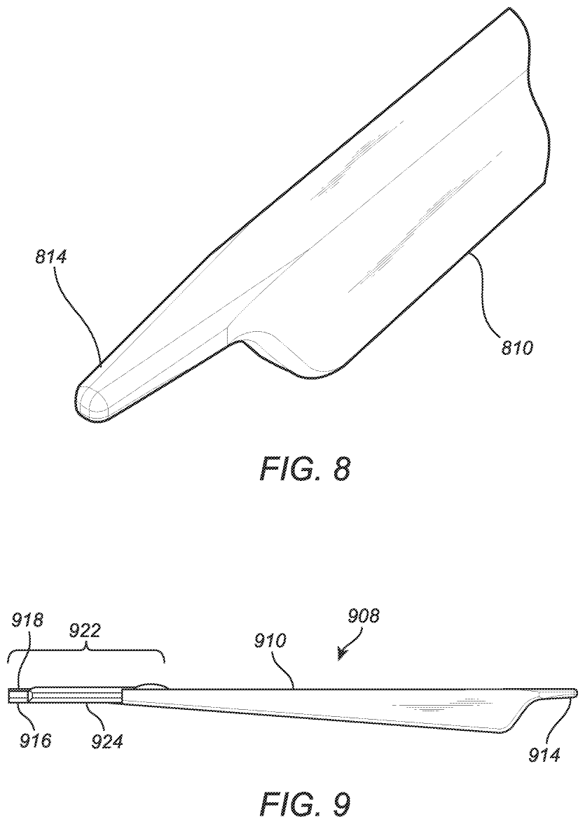

Referring now specifically to FIG. 8, in the second exemplary embodiment, the hook 814 of the present disclosure is shown in greater detail. The hook 814 is disposed on one end of the elongated shaft 810 and tapered therein from the material from which the elongated shaft 810 is manufactured. The hook 814 is extended in the same direction as the elongated shaft 810 and tapers to a rounded point. It should be noted that the hook 814 may be manufactured to form a hook shape or other geometry to perform the desired task. The distal end of said hook 814 is rounded as to allow it to glide across the top of the flexor tendon 104 without causing damage after the A1 pulley 106 is severed, allowing it to be used to confirm the complete release of the A1 pulley 106.

Referring now specifically to FIG. 9, in the second exemplary embodiment, the surgical tool 908 of the present disclosure again includes an elongated shaft 910, a blade assembly 922, and a hook 914. The elongated shaft 910 again is made up of two ends, the blade assembly 922 and hook 914, both fixed to opposite ends of the elongated shaft 910. In this embodiment, the blade assembly 922 again includes a cutting edge 916, a protective spine 918, and a blade cover 924 shown in its retracted position.

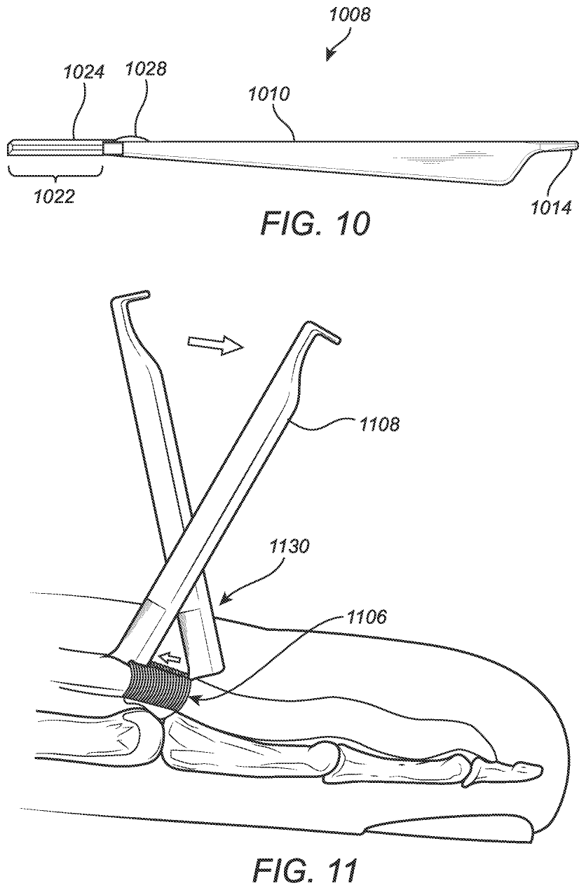

Referring now specifically to FIG. 10, in the second exemplary embodiment, the surgical tool 1008 of the present disclosure again includes an elongated shaft 1010, a blade assembly 1022, and a hook 1014. The elongated shaft 1010 again is made up of two ends, the blade assembly 1022 and hook 1014, both fixed to opposite ends of the elongated shaft 1010. In this embodiment, the blade assembly 1022 again includes a cutting edge 916, a protective spine 918, and a blade cover 1024 shown in its extended position. As before, the blade cover 1024 includes a recess that conforms to the cutting edge 916 and protective spine 918, such that the blade cover 1024 may slide along the length of the blade assembly 1022 when shifted by the user. The blade cover 1024 also includes a raised toggle 1028 which allows the user to push and pull the blade cover 1024 into the desired extended or retracted position. The blade cover 1024 is limited in linear motion by the structure of the elongated shaft or any other type and/or number of mechanisms, such as detents or the like, may also be used to selectively grade and retain the motion of the blade cover 1024 atop the blade assembly 1022. In this illustrative embodiment the blade cover 1024 is illustrated in its extended position to show how it covers the blade assembly. When the blade cover 1024 is fully extended, the entire blade assembly 1022 is hidden inside the blade cover 1024, thus protecting the internal structures of the finger from the cutting edge 916.

Referring now specifically to FIG. 11, the surgical tool of the present disclosure is shown performing the percutaneous A1 pulley release. The surgical tool 1108 is positioned into a small incision 1130 made in the skin centered over the middle of the A1 pulley 1106. It shall be known to one of ordinary skill in the art that the small incision 1130 may be made more proximal or distal instead of in the middle of the A1 pulley 1106. The blade 212 is inserted into the small incision to engage the distal end of the A1 pulley 1106. Alternatively, the blade cover 1024 is retracted prior to inserting the blade assembly 622 into the small incision 1130 and engaging the A1 pulley 1106 with the cutting edge 616 The blade 212 or cutting edge 616 is then rotated proximally to cut the A1 pulley. It should be known to one of ordinary skill in the art that the blade 212 or cutting edge 616 may alternatively engage the proximal end of the A1 pulley 1106 and be rotated distally to cut the A1 pulley 1106.

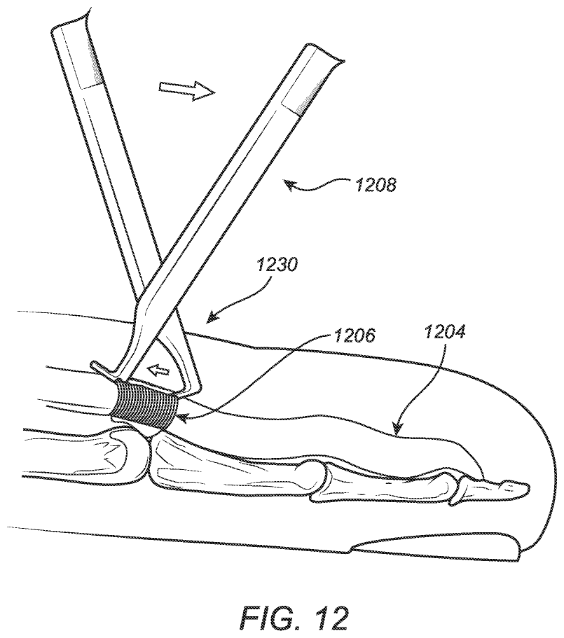

Referring now specifically to FIG. 12, the surgical tool of the present disclosure is shown confirming the percutaneous A1 pulley release. As stated before, the surgical tool 1208 is positioned into a small incision 1230 made in the skin centered over the middle of the A1 pulley 1206. It shall be known to one of ordinary skill in the art that the small incision 1230 may be made more proximal or distal instead of in the middle of the A1 pulley 1206. The hook (214, 414) is inserted into the small incision to engage the distal end of the previously cut A1 pulley 1206. The hook is then translated proximally across the flexor tendon 1204 to confirm the separation of the A1 pulley 1206. Again, it should be known to one of ordinary skill in the art that the hook (214, 414) may alternatively engage the proximal end of the A1 pulley 1206 and be translated distally to confirm the release of the A1 pulley 1206.

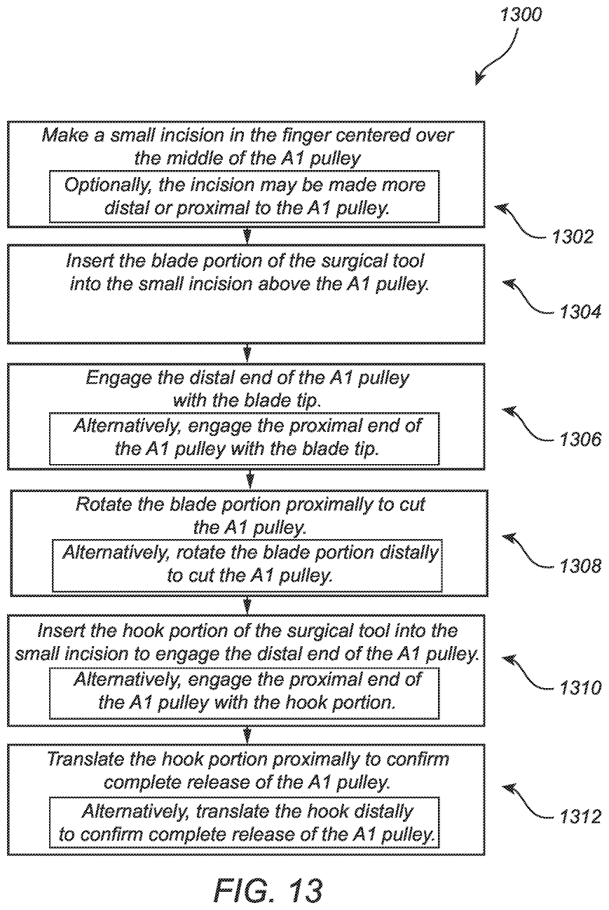

Referring now specifically to FIG. 13, the method 1300 of using the surgical tool of the first embodiment is described. A small incision is made in the skin of the finger as a means of access to the A1 pulley. This incision can be made centered over the A1 pulley or may be made more proximal or distal to the A1 pulley. The blade assembly of the present disclosure is then inserted into the small incision and the blade tip is used to engage either the distal end or the proximal end of the A1 pulley. The blade is then translated either distally or proximally depending on the prior alignment to cut through the A1 pulley and release it. The blade is then removed from the incision and the tool is flipped around to allow the hook to be inserted into the incision. The hook is then used to again engage the distal or proximal end of the released A1 pulley and is translated back and forth to confirm the complete release of the A1 pulley.

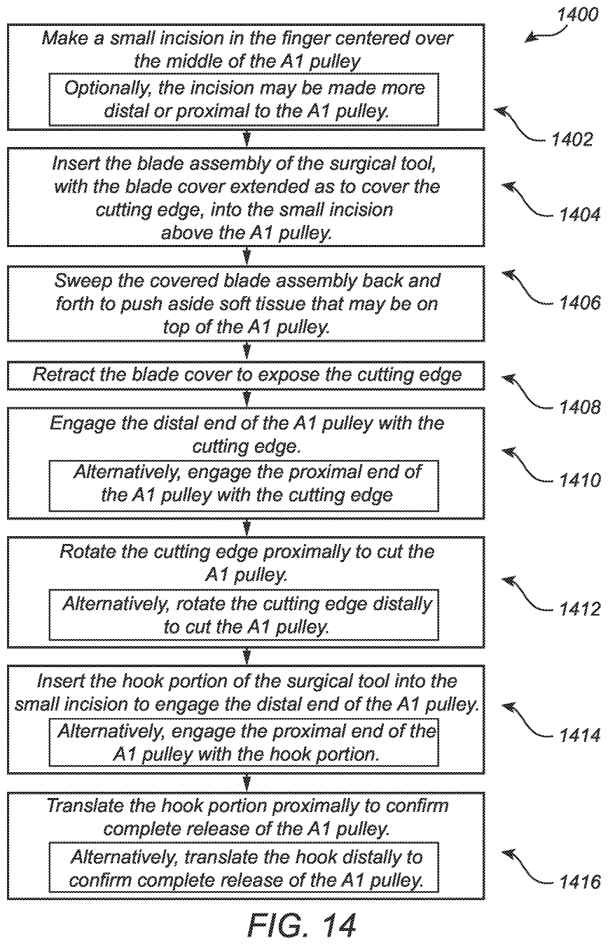

Referring now specifically to FIG. 14, the method 1400 of using the surgical tool of the second embodiment is described. A small incision is made in the skin of the finger as a means of access to the A1 pulley. This incision can be made centered over the A1 pulley or may be made more proximal or distal to the A1 pulley. The blade assembly of the present disclosure is then inserted into the small incision with the blade cover extended as to cover the cutting edge. The covered blade assembly is then used to push aside any soft tissue that may be present on top of the A1 pulley. After the soft tissue is pushed away the blade cover is retracted, thus exposing the cutting edge. The cutting edge is then used to engage either the distal end or the proximal end of the A1 pulley. The cutting edge is then translated either distally or proximally depending on the prior alignment to cut through the A1 pulley and release it. The blade is then removed from the incision and the tool is flipped around to allow the hook to be inserted into the incision. The hook is then used to again engage the distal or proximal end of the released A1 pulley and is translated back and forth to confirm the complete release of the A1 pulley.

Although the present invention is illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A surgical tool, comprising:
an elongated shaft;
a blade assembly coupled to a first end of the elongated shaft; and
a hook coupled to a second end of the elongated shaft.

2. The surgical tool of claim 1, wherein the blade assembly comprises a cutting edge and a protective spine disposed on opposite sides of said blade assembly.

3. The surgical tool of claim 2, wherein the cutting edge further comprises an elongated blade tip protruding from a distal end of the cutting edge.

4. The surgical tool of claim 1, wherein the blade assembly comprises a cutting edge, a protective spine, and a blade cover.

5. The surgical tool of claim 4, wherein the blade cover comprises a raised toggle and a recess that conforms to the cutting edge and protective spine, such that the blade cover is secured to the blade assembly and may slide linearly along the length of the blade assembly to selectively allow the cutting edge to be exposed.

6. The surgical tool of claim 1, wherein the hook is tapered from the elongated shaft and extends perpendicular to the elongated shaft, terminating in a rounded end to protect anatomical structures.

7. The surgical tool of claim 1, wherein the hook is tapered from the elongated shaft, terminating in a rounded end to protect anatomical structures.

8. A method for using a surgical tool, comprising:
Making an incision in skin centered over a middle portion of an A1 pulley in a finger;
Inserting a blade assembly of a surgical tool into the incision;
Engaging a distal end of the A1 pulley with a blade tip extending from a distal end of a cutting edge fixed to the blade assembly;
Translating the blade assembly proximally to cut the A1 pulley;
Removing the blade assembly from the incision;
Inserting a hook of the surgical tool to engage the distal end of the A1 pulley; and
Translating the hook proximally as to confirm the complete release of the A1 pulley.

9. The method for using a surgical tool of claim 8, wherein the incision is made proximal or distal to the A1 pulley.

10. The method for using a surgical tool of claim 8, wherein the blade tip engages a proximal end of the A1 pulley.

11. The method for using a surgical tool of claim 10, wherein the blade assembly is Translated distally to cut the A1 pulley.

12. The method for using a surgical tool of claim 8, wherein the hook engages a proximal end of the A1 pulley.

13. The method for using a surgical tool of claim 12, wherein the hook is translated distally as to confirm the complete release of the A1 pulley.

14. A method for using a surgical tool, comprising:
Making an incision in skin centered over a middle portion of an A1 pulley in a finger;
Inserting a blade assembly of the surgical tool into the incision with a blade cover in its extended position as to cover a cutting edge;
Sweeping the covered blade assembly back and forth to push aside soft tissue that may be on top of the A1 pulley to allow subsequent cutting of the A1 pulley;
Retracting said blade cover as to expose the cutting edge of the blade assembly to allow subsequent cutting of the A1 pulley;
Engaging a distal end of the A1 pulley with the cutting edge of the blade assembly;
Translating the cutting edge proximally to cut the A1 pulley;
Removing the blade assembly from the incision;
Inserting a hook of the surgical tool to engage the distal end of the A1 pulley; and
Translating the hook proximally as to confirm the complete release of the A1 pulley.

15. The method for using a surgical tool of claim 14, wherein the incision is made proximal or distal to the A1 pulley.

16. The method for using a surgical tool of claim 14, wherein the cutting edge engages a proximal end of the A1 pulley.

17. The method for using a surgical tool of claim 16, wherein the cutting edge is translated distally to cut the A1 pulley.

18. The method for using a surgical tool of claim 14, wherein the hook engages a proximal end of the A1 pulley.

19. The method for using a surgical tool of claim 18, wherein the hook is translated distally as to confirm the complete release of the A1 pulley.
